(19) United States
(12) Patent Application Publication
(10) Pub. No.: US 2010/0099630 A1
(43) Pub. Date: Apr. 22, 2010

(54) Method for improving ventilatory efficiency

(76) Inventors: Dean J. MacCarter, Englewood, CO (US); John A. St. Cyr, Coon Rapids, MN (US)

Correspondence Address: Kathleen R. Terry, 13840 Johnson Street NE, Ham Lake, MN, 55304, US

(21) Appl. No.: 11/639,476

(22) Filed: Dec. 15, 2006

Related U.S. Application Data
(63) Continuation-in-part of application No. 11/118,613, filed on Apr. 29, 2005.
(60) Provisional application No. 60/566,584, filed on Apr. 29, 2004.
(60) Provisional application No. 60/608,320, filed on Sep. 9, 2004.

(51) Int. Cl. A61K 31/7004; A61P 11/00

(52) U.S. Cl. 514/23

(57) ABSTRACT

This invention is a method of improving the function of the pulmonary arm of the cardiac-pulmonary axis by the administration of a pharmaceutical or nutritional supplement to a patient in which the function of the pulmonary arm is suboptimal, but not as a sequella of dysfunction of the cardiac arm. The exemplar patient is one suffering from chronic obstructive pulmonary disease. The preferred pentose is D-ribose, to be administered chronically.

### RELATED APPLICATIONS

This application is a continuation-in-part of U.S. patent application Ser. No. 11/118,613, filed Apr. 29, 2005, which claims priority of U.S. Provisional Patent Application Ser. No. 60/566,584, filed Apr. 29, 2004 and Ser. No. 60/608,320, filed Sep. 9, 2004.

### FIELD OF THE INVENTION

This invention pertains to the use of pharmaceutical or nutritional supplements to improve the function of the cardiac-pulmonary axis in those patients in which the function of the cardiac-pulmonary axis is suboptimal.

### BACKGROUND

The cardiac and pulmonary organ systems are closely and inexorably linked, physically and physiologically. Any abnormal physiological change or medical lesion in either arm has a combined and separate impact on these organ systems. This union describes the cardiac-pulmonary axis. The axis contains a pump. The right and left ventricles reside in a closed circuit. The pump fills passively. The pressure stroke which empties the ventricle is termed systole, while the passive filling stage is termed diastole. The right ventricle of the heart is connected to vascular channels: the blood from the right ventricle flows through the pulmonary arteries into the lungs and back to the left atrium and thence to the left ventricle. The blood from the left ventricle through the systemic capillary beds and back to the right side of the cardio-vascular circuit. The efficiency of ventricular action is dependent not only on the condition of the ventricle itself, but on the resistance against which it must pump. This resistance depends on several factors, including the elasticity of the vessels through which blood flows, the compliance of the ventricles for passive filling, circulatory volume, heart rate and the viscosity of the blood.

Changes in any one of these factors within in the circulatory pathway has an impact on the cardiac-pulmonary axis. Loss of elasticity of the blood vessels, for example, due to age-related vascular disease leads to increased resistance against the pumping ventricle. Loss of compliance of the ventricles leads to lower levels of passive filling, with subsequent reduced output. Chronically, increase in the work load on the right ventricle causes the cardiac muscle to increase in size to compensate for the increased demand. Coupled with poor compliance, the function of the right ventricle in perfusing the lungs is compromised. Further, with myocardial cellular tissue dysfunction, pumping efficiency is reduced. Further, an increase in blood viscosity, such as in polycythemia vera, raises the resistance in the vascular channels. Whatever the cause, the feedback loop of the axis eventually presents with reduction in ventilatory efficiency, ventricular compliance, right ventricular hypertrophy, right side heart failure with potential death. Neurological and hormonal components also interplay in this scheme to help maintain homeostasis of the axis, or in regulation of any existing conditions.

Much past attention has been dedicated to therapies to improve the cardiac arm of the cardiac-pulmonary axis, with less attention paid to improving the function of the pulmonary arm. A leading cause of poor pulmonary function is smoking. Individuals with a history of smoking often develop shortness of breath, leading to emphysema, in which the alveoli break down, possibly due to the toxins in tobacco smoke. Notably, smokers have more frequent bronchial and pneumatic infections with potential scarring, all of which lead to chronic obstructive pulmonary disease, with a symptom of “breathlessness” during exercise and sometimes at rest.

Many subjects have sub-optimal pulmonary function as measured in terms of ventilatory efficiency, which leads to fatigue and a poor quality of life. Ventilatory efficiency is defined as the volume of ventilation per unit of CO₂ production reflecting the ratio between breathing and effective perfusion of O₂ and elimination of CO₂ through expired air. It is commonly expressed as the linear slope of VE to VCO₂, VCO₂, being on the x-axis. Included in the group with reduced ventilatory efficiency are those suffering from pulmonary conditions such as emphysema, cystic fibrosis, pulmonary fibrosis, chronic obstructive pulmonary disease, asthma and bronchitis. Even subjects with “normal” lungs can have poor pulmonary function for a variety of reasons. Persons with anemia or low O₂/CO₂ carrying capacity breathe rapidly but ineffectively. Renal disease and exposure to high or low atmospheric pressure may also interfere with pulmonary function. Persons having reduced lung volume from scoliosis, spondylitis, surgery or trauma also do not maintain an optimal ventilation-to-perfusion ratio. Persons suffering from lung cancer often have both anemia and reduced lung volume due to tumors blocking portions of the bronchial tree. A very large cohort of subjects with reduced pulmonary function is those suffering from cardiovascular disease, including patients with stable coronary artery disease, myocardial hypertrophy, hypoplastic lung, cardiomegaly, CHF or congenital heart anomalies.

In the past, pulmonary function was estimated by measuring percent oxygen saturation of the blood, or the kinetics of oxygen uptake (VO₂). While useful, these measurements are an isolated snapshot of a point in time; useful to describe the state of the patient's pulmonary function under the testing conditions, but not able to predict function under differing conditions. A person at rest with normal oxygen saturation or uptake may encounter dyspnea under, for example, exercise conditions, when oxygen demand is higher or under lower oxygen tension, when oxygen availability is lower. Ventilatory efficiency (VE), on the other hand, reflects the actual condition of the lungs, when measured during exercise. (Principles of Exercise Testing and Interpretation, Fourth Edition, Wasserman, K.; Hansen, J. E.; Sue, D. Y; Stringer, W. W.; Whipp, B. J. Lippincott Williams & Wilkins, Philadelphia. Pages 92-96. These teachings are incorporated by reference.)

There exists a deficiency spectrum in ventilatory efficiency. Patients may present with reduced VE even before the diagnosis of a medical condition. These patients may include those with primary lung dysfunction because of emphysema, whether due to smoking or to genetic causes, pulmonary hypertension, asthma, chronic bronchitis and chronic obstructive pulmonary disorders. Patients with autoimmune diseases such as rheumatoid arthritis often develop “rheumatoid lung.” Patients with low lung volume due to premature birth, scoliosis, spondylitis or subdevelopment due to lifelong inactivity also are at risk for early pulmonary complications. Often, persons who consider themselves to be in good health with a good nutritional status are actually somewhat suboptimal in both parameters, rendering them at risk for developing medical conditions or predisposing them to fatigue. Those who would benefit from exercise are disinclined to do so.

An advanced approach to treat and prevent pulmonary dysfunction is to recommend supplementation of key nutrients that will aid healing and enhance the physiological state. Such nutritional formulations may be termed “dietary supplements,” “functional foods” or “medical foods.” in order to formulate an effective dietary supplement or functional or medical food, an understanding of the scientific basis behind the key ingredients is essential. Once a well-grounded recommendation toward dietary modification is made, it may have a powerful influence on delay of onset of a medical condition, slowing of progression of the illness, hastening the recovery and continued maintenance of improved health in the individual afflicted with the medical condition. It would be especially useful to develop a method to identify pulmonary dysfunction from a functional standpoint during the course of disease, even before the patient is aware of his pulmonary dysfunction.

Copending patent application Ser. No. 11/118,613, filed Apr. 29, 2005, discloses a method for treating those patients whose dysfunction of the cardiac arm has progressed to involvement of the pulmonary arm as measured by ventilatory efficiency. The method comprises the treatment with a medical food, D-ribose. Since both arms of the axis are compromised, it is unclear which or both arms are benefited.

No such supplement has been identified to improve the pulmonary arm of the cardiac-pulmonary axis. The need remains to provide a supplement to improve the pulmonary condition of persons suffering from reduced pulmonary function. The need also remains for a therapy to improve the homeostasis of the cardiac-pulmonary axis and to limit the progression of pulmonary dysfunction, whether congenital, primary or acquired.

### SUMMARY OF THE INVENTION

The present invention relates to a method for supplementing the diet of subjects having reduced pulmonary function, or who are at risk of pulmonary dysfunction, which has not yet progressed to cardiac involvement. Supplementation is continued longterm, that is, chronically.

According to the methods of this invention, an effective amount of a pentose is administered to a patient with reduced pulmonary function. The pentose may be D-ribose, ribulose, xylulose or the pentose-related alcohol xylitol (all of which are meant to be included in the term “ribose”). The effective amount of pentose is 0.5 to 40 grams of ribose per day and the preferred effective amount is two to 15 grams per day. The most beneficial regimen is the daily dose administered in at least two to four portions. Any dose of D-ribose will show beneficial effect, but the lower doses must be administered more times per day for maximal effect. Higher daily doses must be divided into several doses, each not exceeding eight grams, in order to avoid gastrointestinal side effects. It has been found that patient compliance is best with a dose of three to eight, preferably five, grams of D-ribose given three times a day. It is most convenient to administer ribose at meals, for example, sprinkled on cereal or salad or added to any cold liquid. The unit dosage may be dissolved in a suitable amount of liquid or may be ingested as a powder.

The above regimen is designed for human subjects. The effective dose for other mammals is dependent on the size of the animal. For a horse, a unit dosage of 50 to 300 grams of ribose is effective. For a dog, an effective dose is 500 mg to three grams of ribose.

### BRIEF DESCRIPTION OF THE DRAWINGS

FIG. 1 shows respiratory rate (RR) versus tidal volume (VT) before (1A) and after (1B) eight weeks of ribose supplementation.

FIG. 2 shows VT versus VE before and after eight weeks of ribose supplementation.

FIG. 3 shows energy expenditure before and after eight weeks of ribose supplementation.

### DETAILED DESCRIPTION OF THE INVENTION

The invention comprises a method for the administration of pentose to a mammal suffering from suboptimal function of the cardiac-pulmonary axis wherein the nidus of the dysfunction resides in the pulmonary circuit or arm. A preferred mammal is one suffering from pulmonary dysfunction, whether congenital or acquired. The pulmonary dysfunction may be mild or severe to life-threatening, sporadic or chronic. A chosen exemplar is a mammal suffering from chronic obstructive pulmonary disease that does not yet involve the cardiac arm. Humans, horses and racing dogs are examples of mammals presenting with suboptimal function of the cardiac-pulmonary axis. Humans generally represent chronic dysfunction while horses and dogs experience sporadic dysfunction following a strenuous race or workout. Race horses often have “hemorrhagic lung” due to extreme exertion, which leads to pulmonary dysfunction and often right ventricular hypertrophy. When the mammal experiencing pulmonary dysfunction is a horse, suitable adjustments must be made in the effective dosage. The preferred effective amount of ribose for a horse is 30 to 250 grams of ribose per day. A tolerable single dosage for horses is 30 to 80 grams of ribose. Racing dogs range in size from the whippet at 35 pounds to the greyhound at 65 pounds. The preferred effective dose for a dog is 0.5 to 20 grams of ribose a day. A single tolerable dosage for a dog is 0.5 to 4 grams of ribose.

D-ribose is a natural 5-carbon sugar found in every cell of the body. It has been found in other studies that the pentoses ribulose, xylulose and the pentose-related alcohol xylitol have effects similar to those of D-ribose; therefore, the subsequent use of the term “ribose” in this application is meant to include D-ribose and these other pentoses. Ribose is the key ingredient in the compositions described in this invention. Other energy enhancers might be included that may augment the effect of ribose. Supplements that act by other mechanisms can be energy enhancers that would optimize the nutritional composition. For example, increasing a vessel's diameter by a vasodilator such as adenosine or nitrate would increase blood flow to hibernating muscle tissue beds and thus improve the transport of ribose and nutrients to that tissue with subsequent positive enhancement of its physiological function.

The effective amount of ribose is 0.5 to 40 grams D-ribose per day and the preferred effective amount is two to 15 grams per day. The most beneficial regimen is the daily dose administered in at least two to four portions. Any dose of D-ribose will show beneficial effect, but the lower doses must be administered more times per day for maximal effect. Higher daily doses must be divided into several doses, each not exceeding eight grams, in order to avoid gastrointestinal side effects. It has been found that patient compliance is best with a dose of three to eight, preferably five, grams of D-ribose given three times a day. It is most convenient to administer ribose at meals, for example, sprinkled on cereal or salad or added to any cold liquid.

The following examples are provided for illustrative purposes only and do not limit the scope of the appended claims.

### Example 1

### Ventilatory Efficiency in CHF

Ventilatory efficiency has been critically shown to be the most powerful, independent predictor of CHF patient survival. Ventilatory efficiency (VE) is determined by the linear, submax relationship between Minute Ventilation (V) and carbon dioxide output (V_{CO2}), V being on the “y axis” and the linear slope being determined using the linear regression model, y=a+bx, “b” representing the slope. The steeper the slope, the worse the ventilation efficiency of the patient.

Ventilation efficiency represents the degree of sympatho-excitation in the heart disease patient that reflects increased dead space in the lungs and augmented mechanoreceptor “drive” from the skeletal muscles. CHF patients with a VE slope greater than 36.9 have a significantly poorer prognosis for survival, as determined by Kaplan Meier graphics, than those CHF patients with a VE slope lower than 36.9. Recently, it has been found that 35 is a cut-off point to differentiate between survivors and non-survivors of CHF.

Ventilation efficiency correlates with the level of cardiac preload or filling pressures to the heart. Higher filling pressures adversely affect pulmonary venous flow and cause pulmonary ventilation-to-perfusion mismatching, thus increasing the ventilatory efficiency slope. Ventilatory efficiency slope has also been shown to correlate inversely with heart rate variability (HRV), a known predictor of sudden cardiac death in CHF patients.

### A. Ventilatory Efficiency During Exercise Testing

As an exemplar cohort of patients with reduced ventilatory efficiency, patients suffering from CHF were recruited. Patients having CHF were selected according to the following criteria:
Male and female 48-84 years of age.
Ejection fraction 30-72%
NY Class III-IV (severe condition).
Test and control groups matched for pre-operative volume status, cardiac medication, measured risk assessment.

The test group was administered D-ribose, 15 grams tid for eight weeks; the controls received 15 grams Dextrose tid. All patients in this group underwent repeated cardiopulmonary exercise using a four-minute sub-maximal step protocol. Patients were tested on a step apparatus. Others in the study were tested on a treadmill with varied grade or on drug-driven exercise simulation for those patients unable to use the other two devices. Symptom-limited peak exercise performance with at least 80-85% of age related maximal heart rate was attempted with each patient. Upper extremity blood pressure was obtained at every two minutes and also at peak exercise.

Patients were tested on a treadmill with varying grade, on a step apparatus or with simulated drug-driven exercise simulation for those patients unable to exercise physically. V_{CO2} and V_{O2max} before and after exercise was measured and VE calculated. The methodology is described in Circulation: www.circulationaha.org Ponikowski et al. Ventilation in Chronic Heart Failure, Feb. 20, 2001, the teachings of which are incorporated by reference. Ventilatory efficiency, VO₂ and O₂ pulse were assessed up to the anaerobic threshold at baseline and again at eight weeks. Weber function class was also determined based on VO₂ at the anaerobic threshold (AT). The results for the first group of test patients (2 females and 13 males) are summarized in Table I. “R” designates D-ribose. Each patient acted as his or her control, that is, results after ribose administration were compared to baseline results. VO₂ efficiency is the O₂ uptake per unit time. O₂ pulse is a measurement of the heart stroke volume.

TABLE I

| Ventilatory efficiency | | VO₂ uptake efficiency | | O₂ pulse | |
|---|---|---|---|---|---|
| Pre-R | Post-R | Pre-R | Post-R | Pre-R | Post-R |
| 50.6 +/− 9.8 | 41.6 +/− 6.4 | 1.00 +/− 0.28 | 1.30 +/− 0.28 | 7.45 +/− 1.8 | 9.04 +/− 1.9 |

| | | |
|---|---|---|
| (p < 0.01) | (p < 0.028) | (p < 0.05) |

Results show that the administration of D-ribose improved the VE by about 20% in this study, Note that the improvement in VO₂ was higher, possibly confirming the earlier observation that a “point in time” measurement alone may not be fully descriptive of pulmonary function. It was also found that several of the patients were reclassified into a higher, that is, less severe, Weber functional class.

### B. Detailed Results of Representative Patients.

A 59 year old male, normal weight, was diagnosed with blockage of the coronary arteries with stable angina, not yet progressing to congestive heart failure. A CAT scan showed no myocardial infarction. Using a treadmill, with incremental increase in grade, his VO₂ max and VCO₂ were determined. Following eight weeks of ribose administration of five grams four times a day, he was retested under the same conditions. Plotting a regression analysis of VO₂ versus log V, the VE slope decreased from 60.2 to 45.5. It is considered that a slope of 36.9 or below indicates impairment of ventilatory efficiency. Therefore, while this patient was not in the normal range of ventilatory efficiency, improvement was marked.

A second patient, a 77 year old male of normal weight, self administered five grams of ribose four times a day for eight weeks. At the beginning of the study, his VE slope was 55.7 following nine minutes of submaximal exercise. At the end of the study, his VE slope had decreased to 45.2. This patient also was tested on the step test. The initial test was rated as “good” and the second test was subjectively considered to be “great.”

A third patient, a 72 year old obese woman, was on nasal oxygen and was tested with drug-driven simulated exercise. After administration of five grams of ribose four times daily for eight weeks, her VE slope decreased from 63.0 to 35.2 and the time of simulated exercise was increased from 7.43 minutes to 11.44 minutes. She was able to discontinue the oxygen. Although her VE was now in the normal range, the test results, although improved were not subjectively rated as “good”.

While these results are encouraging, since these were CHF patients, it is possible that the beneficial pulmonary effect was due to a benefit to the cardiac arm of the axis, an effect that is more fully described in co-pending U.S. patent application Ser. No. 11/118,613, filed Apr. 29, 2005, the teachings of which are incorporated by reference. Little is known of the effect of ribose on the pulmonary arm of patients who are not suffering from cardiac complications.

### Example 2

### Ventilatory Efficiency in Rheumatoid Lung

Autoimmune diseases such as rheumatoid arthritis and sarcoidosis eventually result in poor pulmonary function. Exposure to toxins may cause similar deficits in breathing ability. These conditions are chronic and patients are advised to exercise as much as possible, but many are not willing to do so because of fatigue, shortness of breath and wheezing.

A 53-year old woman developed rheumatoid arthritis in the 1970's. By 1988, she began to show symptoms of rheumatoid lung, began the use of rescue inhalers such as Albuterol® inhaler and was hospitalized for respiratory distress three times in the next five years. At that point, she was prescribed Advair® steroid inhaler, which relieved her symptoms considerably, although she still required a rescue inhaler several times per week. In 2002, she began the administration of ribose, approximately five grams two to three times a day. Within a month, she was able to discontinue the use of the rescue inhaler and to exercise more without breathlessness symptoms.

### Example 3

### Improvement of Ventilatory Efficiency in COPD

Although CHF patients represent a major fraction of the group of patients showing a deficit in ventilatory efficiency as a late sequella of their disease, many patients with normal heart function may also show a deficit in ventilatory efficiency. While the benefit of ribose administration in CHF is disclosed in Example 1, and the improvement of ventilatory efficiency by administration of ribose in patients with pulmonary dysfunction, not suffering from advanced CHF, as shown in Example 2, more information on the effect of ribose on diagnosed primary lung disease was needed before ribose could be recommended for improvement of pulmonary function in those suffering from primary lung dysfunction. It would be most desirable to determine whether progression of the disease can be slowed before involvement of the cardiac arm of the cardiac-pulmonary axis.

A major category of lung disease is chronic obstructive pulmonary disease (COPD). This condition is commonly caused by smoking, however, recurring bouts of bacterial bronchitis in which the pulmonary tissue is attacked by bacteria with inflammation seems to be due to the response to the infection. Among these patients may be tobacco smokers, asthmatics, persons with a genetic absence of alpha-1 antitrypsinogen, industrial or environmental exposure to organic solvents or toxins, or cystic fibrosis.

In order to prevent pulmonary dysfunction at the earliest phase before involvement of the cardiac arm, it is important to identify patterns of measurements, preferably during submaximal exercise (see: Principles of Exercise Testing, supra) that are predictive of the status of the pulmonary arm. The following experiments were designed to identify the useful patterns.

Four patients presenting with chronic obstructive pulmonary disease were tested for various parameters of pulmonary function as described in Example 1. Baseline measurements of pulmonary function were taken during moderate, sub-maximum step exercise. Patients were instructed to self-administer five grams of ribose four times a day. After eight weeks, pulmonary function was again measured during moderate exercise. The results are shown in Table II.

TABLE II

| Patient # | VD/VT | VT/RR | VT/ti | VT_{BTPS} | VCO₂ |
|---|---|---|---|---|---|
| 1: baseline | 0.304 | 0.059 | 1492 | 1.54 | 1.038 |
| Post ribose | 0.253 | 0.065 | 1827 | 1.92 | 1.050 |
| 2: baseline | 0.439 | 0.036 | 779 | 0.830 | 0.460 |
| Post ribose | 0.303 | 0056 | 752 | 1.04 | 0.460 |
| 3: baseline | 0.198 | 0.041 | 373 | 0.614 | 0.280 |
| Post ribose | 0.221 | 0.046 | 706 | 0.873 | 0.454 |
| 4: baseline | 0.475 | 0.013 | 937 | 0.550 | 0.310 |
| Post ribose | 0.280 | 0.018 | 1590 | 0.800 | 0.814 |
| 5. No COPD | | | | | |
| Baseline | 0.212 | 0.090 | 1636 | 1.90 | 0.990 |
| Post ribose | 0.221 | 0.100 | 2232 | 2.40 | 1.26 |

In Table II, the units are:
VD=volume of the dead space; VT=tidal volume in liters per minute. The ratio VD/VT can be expressed as ml/ml. This ratio is taken at the nadir of sub-maximal exercise and is a measure of lung function.
VT=tidal volume in liters; RR=breaths per minute
VT=volume in ml at each inspiration, ti=inspiratory time. VT/ti is the inspiratory drive (effort) in ml/second.
VT=tidal volume in liters at constant body temperature pressure status.
VCO₂=liters/minute of expired CO₂
RR=breaths per minute. VT/RR=tidal volume in liters per breath.

Table II illustrates that no one measurement or ratio is predictive of the clinical state of COPD and response to ribose administration. For example, Patient 1, an asthmatic patient with COPD, shows a pattern shift with improvement in VD/VT. Patient #2, diagnosed with COPD, shows changes in most of the parameters following ribose administration; reduced RR/VT slope; increased VT to VE slope; improved VD/VT ratio and increased energy expenditure at VD/VT nadir. Patient #3 has partially improved VD/VT and VCO₂ patterns. Patients #4 shows dramatic pattern reversal with VD/VT following ribose administration. Patient #5 was included to show that the early-identified patient at risk for COPD could benefit from ribose administration. One goal of this study was to determine whether the progression of pulmonary dysfunction in such a patient could be slowed or halted over time.

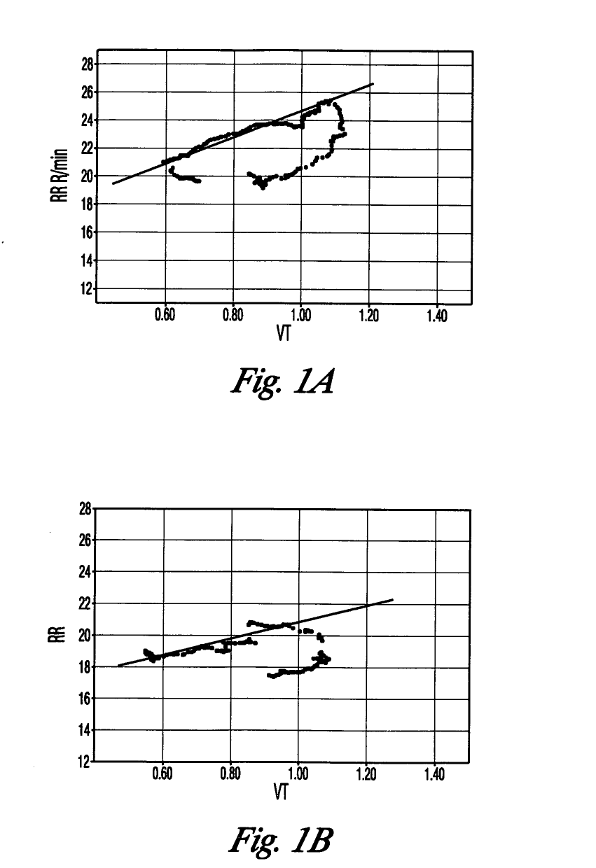

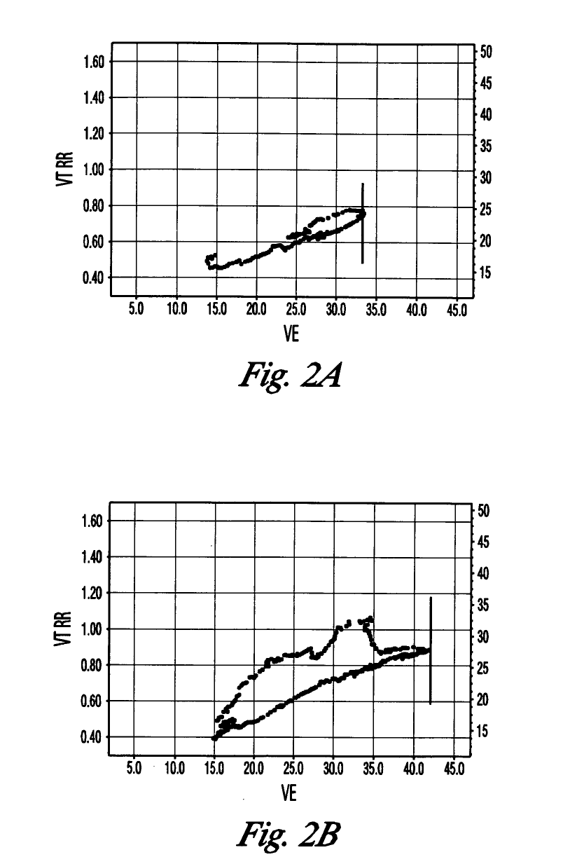

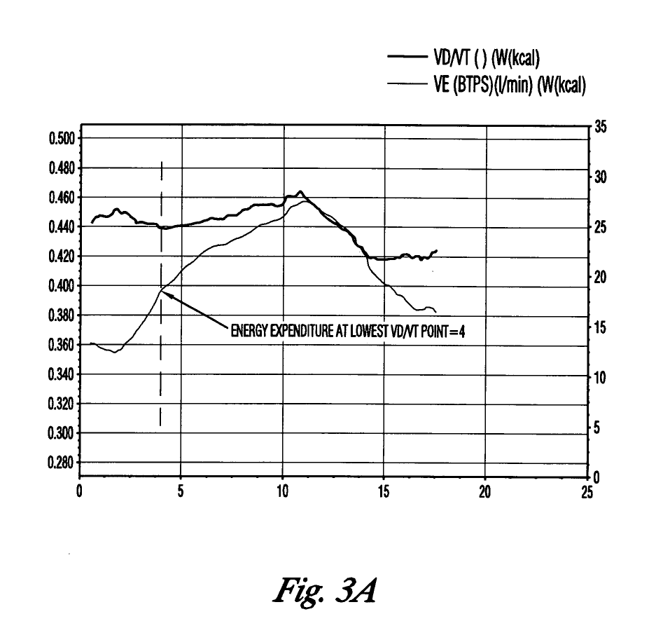

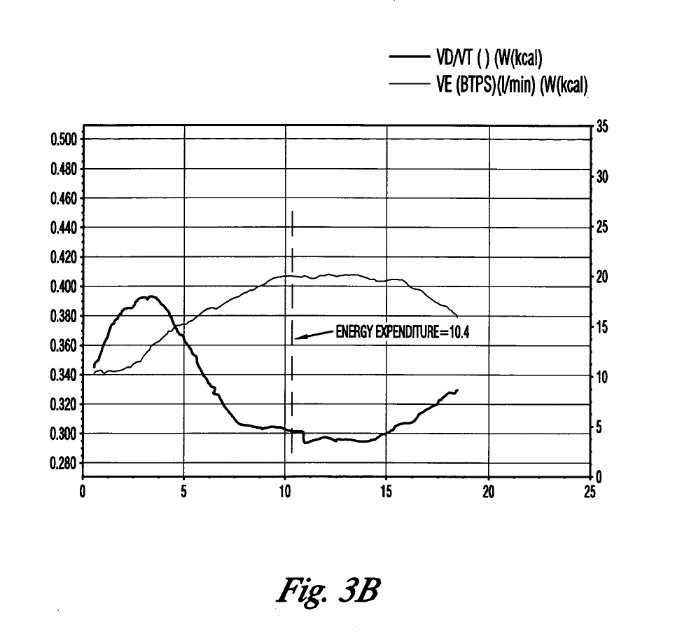

These patterns may be understood better when plotted on a graph. Each figure is based on a single patient and is representative of the various ratios. FIG. 1 shows that when respiratory rate is plotted against tidal volume, ribose administration results in a decreased slope, that is, more efficient breathing. FIG. 2 shows a reduced respiratory rate with elevated VE value of 42 liters/minutes and an increased tidal volume of 0.9 liters as compared to the same values pre-ribose, indicating improved breathing reserve during exercise. FIG. 3 shows the energy expenditure during exercise, pre- and post-ribose.

Overall, review of these pulmonary graph patterns shows that patients with reduced function of the pulmonary arm of the cardiac-pulmonary axis show significantly improved pulmonary performance during exercise by facilitating a reduced dead-space and improving ventilation-to-perfusion matching. Increased tidal volume attained at the nadir of VD/VT ratio appears to aid in gas exchange at the alveolar/capillary membrane interface. In addition, an improvement observed in RR to VT slope may be an indirect measurement of improvement in pulmonary compliance, as well as the observed increase of VT to VE slope.

Data in the table and in the figures demonstrate a more optimal ratio of VT/RR, thus reducing ventilatory work during exercise when ribose is administered. Energy expenditure is actually able to increase at the point of optimal lung performance (FIG. 3). In addition CO₂ production and elimination are shown to increase with ribose administration to patients with reduced pulmonary function, with or without COPD. Regardless of the proposed mechanisms of ribose in patients with reduced pulmonary function, ribose appears to augment lung function, a key component to improving functional capacity. These patients and others should be followed longterm for years to determine whether progression to more serious lung dysfunction and involvement of the cardiac arm of the cardiac-pulmonary axis can be slowed or halted.

All references cited within are hereby incorporated by reference. It will be understood by those skilled in the art that variations and substitutions may be made in the invention without departing from the spirit and scope of this invention as defined in the following claims.

We claim:

1. A method for improving ventilatory efficiency during exercise of a subject comprising the chronic administration of two to ten grams of D-ribose one to four times daily to the subject.

2. The method of claim 1 wherein three to five grams of D-ribose is administered one to four times daily to the subject.

3. The method of claim 1 or 2 wherein D-ribose is administered one to four times daily for at least one week.

4. The method of claim 1 wherein a vasodilator co-administered with D-ribose.

5. The method of claim 5 where the vasodilator is L-arginine, nitroglycerine, a nitrate, a nitrite, papaverine, isoproterenol, nylidrin, isoxsuprine, nitroprusside, adenosine, xanthine, ethyl alcohol, dipyramide, hydrazaline, minoxidil or diazoxide.

6. A method of treating pulmonary dysfunction in a subject who is not suffering from cardiac complications comprising the chronic administration of two to ten grams of D-ribose one to four times daily to the subject.

7. The method of claim 6 wherein the pulmonary dysfunction is chronic obstructive pulmonary disease.

8. The method of claim 6 wherein the pulmonary dysfunction in a subject is caused by exposure to industrial or environmental organic solvents or toxins or tobacco smoke.

9. The method of claim 6 wherein three to five grams of D-ribose is administered three or four times daily to the subject.

10. The method of claim 6 wherein D-ribose is administered one to four times daily for at least one month.
